(19) United States
(12) Patent Application Publication
(10) Pub. No.: US 2024/0034709 A1
(43) Pub. Date: Feb. 1, 2024

(54) NOVEL PROCESS FOR CONTINUOUS PREPARATION OF METHACRYLIC ACID BY CATALYTIC HYDROLYSIS OF METHYL METHACRYLATE

(71) Applicant: Röhm GmbH, Darmstadt (DE)

(72) Inventors: Steffen Krill, Muehltal (DE); Daniel Helmut König, Richmond, TX (US); Dirk Bröll, Langen (DE)

(73) Assignee: Röhm GmbH, Darmstadt (DE)

(21) Appl. No.: 18/550,426

(22) PCT Filed: Mar. 7, 2022
(86) PCT No.: PCT/EP2022/055702

§ 371 Date: Sep. 13, 2023

(30) Foreign Application Priority Data
Mar. 15, 2021 (EP) 21162491.1

(51) Int. Cl. C07C 51/377; C07C 51/44

(52) U.S. Cl. CPC C07C 51/377; C07C 51/44

(57) ABSTRACT

A continuous process can be used for preparing methacrylic acid by catalytic hydrolysis of methyl methacrylate that has been prepared proceeding from C-2, C-3, or C-4 raw materials. In this process, methyl methacrylate of high purity is reacted with water in the presence of a Brønsted catalyst to give a reaction mixture containing the reactants and products and worked up in a distillation column. In the distillation column, a condensate containing an azeotrope of MMA with methanol is produced at the top, a vapour condensate containing methacrylic acid of high purity is produced in the middle part of the column, and a substance mixture containing high-boiling by-products and a small amount of methacrylic acid is obtained in the bottom of the column.

### FIELD OF THE INVENTION

The present invention relates to a continuous process for preparing methacrylic acid by catalytic hydrolysis of methyl methacrylate that has been prepared proceeding from C-2, C-3 or C-4 raw materials. In this process, methyl methacrylate of high purity is reacted with water in the presence of a Brønsted catalyst to give a reaction mixture comprising the reactants and products and worked up in a distillation column, at the top of which a condensate comprising an azeotrope of MMA with methanol is produced, a vapour condensate containing methacrylic acid of high purity is produced in the middle part of the column, and a substance mixture containing high-boiling by-products and a small amount of methacrylic acid is obtained in the bottom of the column. A further aspect of the present invention is the separation of an MMA-water azeotrope from the desired methacrylic acid product, and the recycling thereof into the reaction.

Additionally presented are various embodiments for the efficient continuous preparation of methacrylic acid, especially the integration of azeotrope workup of the MMA-MeOH and MMA-water mixtures from methacrylic acid production with workup sections of the MMA production process, wherein it is optionally possible to recycle reactants such as MMA and/or water into the methacrylic acid production process and/or to use one or more of these azeotropes optionally in the preparation of MMA.

The present invention, compared to prior art processes, permits apparatuses to be dispensed with, and hence lowers capital costs in the building of a new plant. In addition, the present process permits an increase in product yields, combined with a reduction in the level of by-products and the associated cost and inconvenience of disposal, and reduction of the specific energy consumption.

### PRIOR ART

The present invention relates to a novel continuous process for preparing methacrylic acid (MA) based on the hydrolysis of methyl methacrylate (MMA) or other methacrylate esters.

Methacrylic acid is used in large volumes for preparation of polymers and, together with other copolymerizable compounds, in copolymers. For example, methacrylic acid is a constituent of solvent-resistant gloves, can be used in the production of dimensionally stable foams and carbon fibres, and is a basis in formulations for concrete plasticizers (PCEs) and a multitude of further polymers in which MA creates specific properties. In addition, methacrylic acid as a starting material for specialty esters that are produced by esterification with appropriate alcohols. Methacrylic acid is also used for the preparation of hydroxy esters that are constituents of coating and paint formulations.

There is consequently a great interest in very simple, economic and environmentally friendly processes for preparing this important chemical product.

The preparation of MA is based on three possible raw material groups based on C3, C4 or C2 units.

C3 units are the first commercially significant group. MA is predominantly prepared here proceeding from hydrogen cyanide and acetone via the acetone cyanohydrin (ACH) formed as central intermediate. This process has the disadvantage that very large amounts of ammonium sulfate are obtained, the processing of which is associated with very high costs. Further C3-based processes which use a raw material basis other than ACH are described in the relevant patent literature and have now been implemented on a production scale, but have similar problems.

Additionally known are processes for preparing MMA proceeding from methacrylamide (MAm). In this case, the ACH is typically first reacted with sulfuric acid, forming a sulfuric acid solution of MAm after passing through a multistage reaction sequence. This substance mixture is reacted with water, with hydrolysis of MAA to MA to give the resultant ammonia in the form of ammonium hydrogensulfate. A multitude of such processes is described in the prior art, for example in U.S. Pat. No. 7,253,307, according to which MAm is reacted with water at moderate pressures and temperatures between 50° and 210° in the presence of superstoichiometric amounts of sulfuric acid to give methacrylic acid.

The process according to U.S. Pat. No. 7,253,307 intrinsically gives good yields and permits the preparation of a methacrylic acid quality of high purity, but large amounts of ammonium sulfate-containing sulfuric acid wastes arise, which have to be thermally regenerated to give fresh sulfuric acid or else can be disposed of. The separation and isolation complexity for obtaining the methacrylic acid is correspondingly demanding and typically comprises a phase separation and at least two distillative separation steps. In the last separation step, methacrylic acid is obtained as top condensate in commercial purity; the by-products of the reaction are obtained in the bottom of the column, often undefined dimers and oligomers, the formation of which cannot be suppressed by this process.

There are not only known processes for preparing MA that proceed from MAm.

In an alternative process, hydroxyisobutyramide (HIBA) is used as reactant.

Such a process is described in U.S. Pat. No. 3,487,101, by which methacrylic acid itself and methacrylic esters derived therefrom are obtainable. HIBA in the liquid phase is admixed here with sodium hydroxide solution, for example, in the presence of homogeneous basic catalysts, forming HIBA salts as intermediate, from which water can be eliminated at temperatures up to 320° C., and MA is then formed. The MA can then be removed overhead. In the embodiment described here, high-boiling esters, for example dimethyl phthalate or phthalic anhydride, are used as dehydrating agents that additionally serve as solvents for the reaction matrix. Very good selectivities of about 98% coupled with high conversions are described. With regard to the complicated and multistage process for preparing HIBA, it is understandable that this process has not yet become established as a method of production in industry. HIBA can be obtained in two stages by repeated hydrolysis from acetone cyanohydrin, forming hydroxyisobutyramide as an intermediate, which can in turn react further to give the acid. Here too, when sulfuric acid is used as reagent, ammonium sulfate-containing sulfuric acid solutions are formed, the regeneration of which is of unlimited complexity.

Further embodiments and optimizations for performance of the HIBA conversion to MA are disclosed in DE 191367. Catalysts used here are zinc bromide and lithium bromide, which leads to a highly selective reaction. However, the halide-containing catalysts and the high temperatures, on account of the high corrosivity, place extreme demands on the materials of the plant, and the formation of halogenated by-products that are obtained in the distillate with methacrylic acid and would have to be removed in a complex manner mean that the process is not very attractive.

EP 04 873 53 describes a complex multistage process proceeding from acetone cyanohydrin, wherein hydroxyisobutyric acid is likewise used here as reactant for the central step of the preparation of MA. ACH is hydrolysed catalytically in a first reaction step, for example in the presence of heterogeneous manganese dioxide catalysts. Hydroxyisobutyramide (HIBA) is formed in high yield. In the next step, HIBA is reacted with methyl formate or mixtures of methanol/carbon monoxide, giving complex product mixtures containing methyl hydroxyisobutyrate (MHIB) and formamide. Formamide is dehydrated in a separate reaction stage to give hydrogen cyanide, in which case it is again possible subsequently to react HCN with acetone to give ACH. MHIB is hydrolysed in the presence of a heterogeneous acidic ion exchanger with water to form HIBA, which is then reacted catalytically with basic alkali metal salts with elimination of water to give methacrylic acid. The multitude of reaction steps necessary mean that the process is not very attractive, especially with regard to the significant capital costs for the building of a plant of this complexity.

Isobutylene or tert-butanol as C-4-based raw materials are of growing significance nowadays as reactants for preparation of MA. These are converted to MA over several process stages. A third alternative starting material that may also be used is methyl tert-butyl ether (MTBE), which is converted to isobutene by elimination of methanol. In these preparation methods, isobutylene or tert-butanol is oxidized to methacrolein in a first stage, and this methacrolein is then reacted with oxygen to give methacrylic acid. The MA obtained is either isolated and purified or converted to MMA and other esters. More details of this process are given, inter alia, in Ullmann's Encyclopedia of Industrial Chemistry 2012, Wiley-VCH Verlag GmbH & Co. KGaA, Weinheim, Methacrylic Acid and Derivatives, DOI: 10.1002/14356007.a16_441.pub2 and in Trends and Future of Monomer-MMA Technologies, SUMITOMO KAGAKU 2004-II. Further details relating to MMA and methacrylic acid preparation processes in general and specifically to the multistage gas phase process proceeding from C4 units are described in Krill and Rühling et al. “Many paths lead to methacrylic acid methyl ester”, WILEY-VCH Verlag GmbH & Co. KGaA, Weinheim, doi.org/10.1002/ciuz.201900869.

In general, the C4 route starts from the steam cracking product IBEN or alternatively also TBA, which is oxidized to methacrolein (MAL) in the first step by means of gas phase oxidation. In a second gas phase oxidation stage, the MAL obtained as intermediate is oxidized to MA. The gaseous reaction products are cooled down in a downstream quenching step and to a very great extent condensed. It is characteristic of the process that the second reaction stage does not involve complete conversion with respect to MAL and that unconverted MAL is recovered in an absorption and desorption unit (recycle MAL) in order to subsequently feed it back as a feed for the second reaction stage.

Isobutylene or tert-butanol can be reacted with atmospheric oxygen in the gas phase over a heterogeneous catalyst to give MAL and then converted to MMA by means of an oxidative esterification reaction of MAL using methanol. This process is described, inter alia, in U.S. Pat. Nos. 5,969,178 and 7,012,039. The disadvantages of this process relate especially to a high energy demand, one reason for which is the mode of operation at ambient pressure. This process avoids the problem of an evaporation of MAL since the process takes place in the liquid phase, and so MAL does not need to be converted to the gaseous state and the problem of mixing with critical oxygen-containing gases is thus circumvented. It is not possible to use this to derive a solution for optimizing the two-stage isobutene gas phase process with MA as intermediate.

Another problem with all these processes is the relatively unsatisfactory yield, caused in particular by high losses in the oxidation steps and associated formation of CO₂. It must also be pointed out that this is also associated with by-product formation, which entails complex process steps for isolation of the product. For instance, all processes proceeding from isobutylene or equivalent C4-based raw materials such as TBA or MTBE achieve selectivities of 80% to 90% per process stage in the gas-phase oxidation over a heterogeneous catalyst system. Thus, based on the C3 or C4 starting material, an overall yield of not more than 65% to 70% is achieved. By its nature, the gas phase process proceeds at moderate pressures of between 1 and 2 bar absolute and generates a process gas in which the product component is present only at about 4% to 6% by volume. The isolation of the product of value from the inert gas ballast is correspondingly energy-intensive and consumes large amounts of cooling energy and steam for multistage distillative workup steps. What is common to these processes, moreover, is that they are typically conducted in the gas phase in the presence of heterogeneous catalysts. As a result, the separation complexity is additionally considerable, especially as a result of the necessity of removal of the MA, also with regard to the gas ballast.

Ethylene as C2 unit can also be used as raw material for the production of MA.

By reaction of ethylene with carbon monoxide or synthesis gas, it is possible to prepare and isolate propionaldehyde (PA) or propionic acid as a conversion product of PA. The unsaturated carbonyl compounds can be prepared efficiently from these primary intermediates with formalin or formaldehyde by way of an aldolization. Methacrolein is obtained here from PA, and MA directly from propionic acid. Methacrolein in turn can be catalytically oxidized further to MA.

Neither process has become established industrially and commercially, one reason being that the catalyst systems used do not have sufficient long-term stability. In the reaction of propionic acid with formaldehyde in the gas phase, the activity of the gas phase catalysts used falls drastically even after a few hundred hours; one can speculate that this is attributable to significant coking and deposition of nonvolatile substances on the catalyst surface. On the other hand, the oxidation of methacrolein in the presence of water and solvents in the presence of specific precious metal catalysts leads to gradual dissolution of the carrier components, and so the activity of the catalysts cannot be sustained here either.

U.S. Pat. No. 8,791,296 describes a process for preparing methacrylic acid based on the hydrolysis of methacrylic esters, comprising the following process steps: providing acetone cyanohydrin, converting acetone cyanohydrin to methacrylamide, esterifying methacrylamide in the presence of alcohols to give the corresponding methacrylic ester, and hydrolysing the methacrylic ester to methacrylic acid. This process succeeds in preparing methacrylic acid in high purities of ≥99.5%, but the process is limited to the use of acetone cyanohydrin-based MMA preparation processes, and a total of four process steps are needed for the continuation of the process for isolation of methacrylic acid, which entails an elevated energy requirement. The first process step comprises the hydrolysis of methyl methacrylate to methacrylic acid. Subsequently, three distillation steps at different pressures are needed.

A further feature of the process is that large circulation streams from two workup apparatuses have to be recycled to the actual reaction stage, with the ratio of the circulation streams accounting for at least five times the amount based on the feed stream into the reactor. The process comprises a reactor, a rectification column for a removal of methanol, the top condensate from which is recycled into the reactor, and a further rectification column operated under reduced pressure for the removal of low boilers. Methacrylic acid is isolated in pure form and high quality as top product, more specifically as condensate of the top stream, from a third column operated under reduced pressure. The multitude of apparatuses and the high recycling rate of condensates, some of which inhibit the equilibrium reaction, mean that the process is energetically disadvantageous, and operating and capital costs are high.

In summary, there are a multitude of known processes for preparing MA that proceed either from acetone (C3), propylene (C3), ethylene (C2) or isobutene (C4). Central intermediates prepared and isolated here are ACH, isobutyric acid or hydroxyisobutyric acid. Processes that have become established industrially are especially the MA processes that proceed from ACH and isobutene, called the C-3- and C-4-based processes. The established processes are summarized by way of overview in the literature and discussed, for example, in Weissermel, Arpe “Industrielle organische Chemie” “Industrial Organic Chemistry”, VCH, Weinheim 1994, 4th edition, p. 305 ff. or in Kirk Othmer “Encyclopaedia of Chemical Technology’, 3rd edition, vol. 15, page 357.

### Problem

Against the background of the prior art discussed, the problem addressed was that of providing a novel process for preparing methacrylic acid that has the disadvantages of the prior art only to a reduced degree, if at all.

More particularly, the problem addressed by the present invention was that of providing a novel process that can be implemented with a minimum level of apparatus complexity and hence low capital costs. At the same time, general operating costs and specific energy consumption in continuous operation should be kept at a minimum.

A particular aspect to be resolved was additionally the avoidance or reduction of operational faults as a result of polymeric deposits in continuous sustained operation of said plant.

A further problem was that of simplifying the product workup for achievement of an on-spec (meth)acrylic acid quality, and the optimal removal and recycling or physical utilization of MMA-containing azeotropes obtained.

It was a further object of the invention to provide by-products of the MMA hydrolysis in a form that permits simple workup and reliable circulation. This also includes separation of the azeotrope of methanol and methyl (meth)acrylate, and integration into a process for preparing MMA.

Further problems which are not stated explicitly may become apparent from the description of the invention that follows, the claims, the examples or the overall context of the present invention.

### Solution

The stated problems have been solved by the provision of a novel process for continuously preparing (meth)acrylic acid. This novel, continuously performable process is based on the reaction of (meth)acrylic esters, especially of methyl methacrylate, with water in the presence of an acidic catalyst in the form of a catalytic hydrolysis.

This process according to the invention has the following process steps (a) and (b):
(a) In a reactor I, a (meth)acrylic ester and water are converted in the presence of a Brønsted acid. This affords a mixture containing at least one (meth) acrylic ester, water, and an alcohol corresponding to the (meth)acrylic ester and an unsaturated acid.

Thereafter, in process step
(b) this mixture is separated in a rectification column having an upper, middle and lower region.

This rectification column—also referred to hereinafter simply as column—is notable for the following features:
(i) In the upper region of the column, at the top of the column, a mixture consisting of the alcohol and (meth)acrylic ester is removed.
(ii) In a side draw S1 of the column which may, for example, be in the middle region of the column, a mixture of (meth)acrylic ester and water is removed and hence withdrawn.
(iii) In the middle region, in a side draw S2 of the column, which may, for example, be in the middle region of the column, (meth)acrylic acid is removed and withdrawn.
(iv) In the lower region, in the bottom of the column, a substance mixture containing higher-boiling components relative to (meth)acrylic acid is withdrawn.

Preferably, the (meth)acrylic ester is MMA, the alcohol is correspondingly methanol, and the (meth)acrylic acid formed is methacrylic acid.

The term “(meth)acrylic acids” is known in the art and is understood to mean acrylic acid and methacrylic acid. The term “(meth)acrylic esters” is known in the art and is understood to mean acrylic esters and methacrylic esters.

However, the process is also applicable, with slight modifications that are easy for the person skilled in the art to derive in a specific manner, to other alkyl (meth)acrylate as well, such as butyl (meth)acrylate or ethylhexyl methacrylate in particular. It is even possible to apply the process to functionalized (meth)acrylates such as hydroxyethyl methacrylate.

### Reactor I

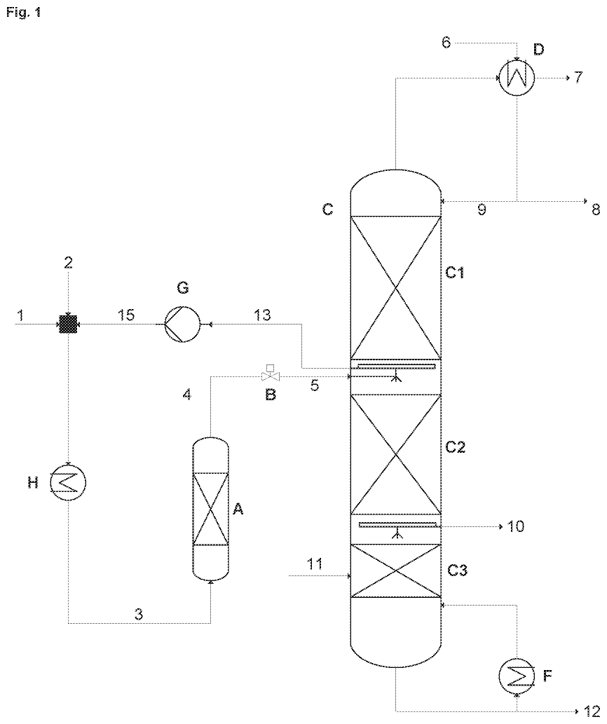

The process, as described, has a reactor I in which at least one catalyst is preferably provided. This reactor I need not necessarily be a reactor operated in isolation. Instead, reactor I may also take the form of a reaction region. Reactor I here may be inside and/or outside the rectification column. However, this reactor is preferably implemented outside the rectification column in a separate region, this being shown in detail for preferred embodiments in FIGS. 1, 2 and 3. Flow tube reactors have been found to be particularly favourable for such a separate reactor I.

The following process parameters are particularly favourable for the reaction in reactor I:

The reaction is generally conducted preferably at temperatures in the range from 20° C. to 200° C., more preferably at 40 to 150° C., especially at 60 to 110° C. The reaction temperature here depends on the system pressure established.

In the preparation of methacrylic acid from methyl methacrylate and water, the reaction temperature is preferably 60 to 130° C., more preferably 70 to 120° C. and most preferably 80 to 110°C.

With regard to the operating pressure, which indirectly also determines the reaction temperature, a distinction is made in terms of the exact execution of the present invention. In the case of an arrangement of the reactor inside the column, the reaction is preferably performed within the pressure range from 5 to 200 mbar, especially at 10 to 100 mbar and more preferably at 20 to 50 mbar.

If the reactor is outside the column, different pressure and temperature conditions from those in the column may be chosen therein. This has the advantage that the reaction parameters of the reactor may be adjusted independently of the operating conditions in the column. If the reactor is outside the column, the reaction is preferably conducted at pressures in the range from 0.5 to 20 bar, more preferably at 1 to 10 bar, especially preferably at 3 to 5 bar.

All pressures given are absolute pressure figures.

The reaction time of the reaction depends on the reaction temperature; the residence time in the reactor for a single pass is preferably 0.5 to 15 minutes and more preferably 1 to 5 minutes.

Preference is given to performing the process according to the invention in such a way that reactor I is supplied continuously with a reactant mixture of (meth)acrylic ester and water in a molar ratio between 1:20 and 20:1.

In the specific preparation of methacrylic acid from methyl methacrylate and water, the molar feed ratio of water to methyl methacrylate is preferably 0.5 to 20:1, more preferably 0.5 to 10:1 and most preferably 1.0 to 4:1.

The reaction mixture may, as well as the reactants, comprise further constituents, for example solvents, catalysts and polymerization inhibitors.

### The Rectification Column

With the aid of the column used in accordance with the invention, having the separation sections described, the methacrylic acid is surprisingly removed in high purity in the middle section of the column in a very simple manner and with a low level of complexity. The rectification column may be produced here from any material suitable therefor. Suitable materials for this purpose include stainless steel and other suitable inert materials.

Preference is given to an execution of the present invention in which the (meth)acrylic ester and water starting materials present in the side draw S1 are recycled into the reaction region of the reactor I. These starting materials are reacted therein with fresh water and (meth)acrylic ester. Optionally, the side draw stream is subjected to a phase separation before being at least partly recycled in the reaction.

It is particularly advantageous when the column used in accordance with the invention is configured in such a way that the (meth)acrylic acid is removed already in a purity greater than 95% by weight via side draw S2. The side draw S2 here is generally beneath the side draw S1 and the feed stream in the column.

The pressure at the top of the rectification column used according to the present invention is preferably 5 to 1200 mbar, more preferably 20 to 1100 mbar and most preferably 50 to 500 mbar. The top stream obtained, after withdrawal from the column, is preferably subjected to a further separation of matter in order to obtain remaining methyl (meth)acrylate and the corresponding alcohol, especially methyl methacrylate and methanol, separately from one another. In this way, it is possible to recycle purified, incompletely converted methyl (meth)acrylate back into the process to increase the yield.

It is possible to discharge high boilers such as added inhibitors by customary methods from the bottom of the rectification column used according to the present invention. This can be effected, for example, with the aid of a thin-film evaporator or a corresponding alternative device. The isolated evaporating substances are more preferably recycled into the rectification column, and non-evaporating high boilers are discharged.

For the reaction according to the present invention, for example, it is possible to use a rectification column having 5 to 20 separating plates each in the upper, middle and lower region. More preferably, the number of separating plates in the upper region is 5 to 15, and 5 to 15 in each of the middle and lower regions. In the present invention, the number of separating plates is understood to mean the number of trays in a tray column multiplied by the tray efficiency, or the number of theoretical plates in the case of a packed column or a column having random packing.

Examples of the rectification column having trays include those such as bubble-cap trays, sieve trays, tunnel-cap trays, valve trays, slotted trays, slotted sieve trays, bubble-cap sieve trays, nozzle trays, centrifugal trays; suitable random packings for a rectification column having random packings are industrially available random packings corresponding to the prior art. Examples are the Raschig Super-Ring or the Sulzer NeXRing. Suitable structured packing includes industrially available metallic structured packings, for example MellapakPlus (Sulzer) or the RMP structured packing from RVT. Additionally usable are structured packings having catalyst pockets, for example Katapak (Sulzer).

A rectification column having combinations of regions of trays, of regions of random packings and/or of regions of structured packings may likewise be used.

Preference is given to using a rectification column having random packings and/or structured packings for the 3 regions. Particular preference is given to using internals that lead to low pressure drops in operation according to the invention.

According to the embodiment and operating parameters, there exist a multitude of industrially used types of collector and distributor. For example, chimney tray collectors are particularly suitable for the complete withdrawal of liquid side streams. Pipe distributors enable high distribution densities, for example, and can reduce inhomogeneous liquid distributions and hence reduce the risk of polymerization.

An example of an illustrative execution is as follows: The feed streams of the fresh reactants are preferably fed into reactor I with the recycle stream that consists predominantly of unconverted reactants and has been obtained from the column. There may be an inert boiling oil in the bottom of the column in order to prevent long dwell times of the (meth)acrylic acid target product. (Meth)acrylic acid is drawn off, preferably in gaseous form, between the middle and lower regions, while the methanol formed is drawn off at the top of the column as an azeotrope with methyl (meth)acrylate and traces of water as the lowest-boiling reaction component. Unconverted reactants are recycled into the reaction region, for example by means of a pump.

### The Catalyst

Preference is given to using heterogeneous catalysts in reactor I, including in an embodiment of a reaction region within the column. Particularly suitable heterogeneous catalysts are acidic fixed bed catalysts, in particular acidic ion exchangers.

Particularly suitable acidic ion exchangers especially include cation exchange resins, such as styrene-divinylbenzene polymers containing sulfonic acid groups. Suitable cation exchange resins are commercially available under the Amberlyst® trade name, under the Dowex® trade name and under the Lewatit® trade name.

A heterogeneous fixed bed catalyst may be used in any region of the rectification column. This is preferably used in the middle region of the column.

The amount of catalyst in litres is preferably 1/10 to 10 times, more preferably ⅕ to 5 times, the amount of newly formed (meth)acrylic acid to be produced in l/h.

The amount of catalyst reported in litres in the feed to reactor I is in a ratio to the amount of (meth)acrylic acid measured in litres which is withdrawn from the column via side draw S2 of 1:10 to 10:1, preferably between 1:5 and 5:1.

In addition, the catalyst may be provided in a separate region of reactor I, in which case this region is connected to the further regions of the apparatus. This separate arrangement of the catalyst region is preferred, it being possible to pass the reactants constantly through the catalyst region. This results in continuous formation of (meth)acrylic acid, and newly formed methanol.

An alternative embodiment is the use of a homogeneous catalyst, for example sulfuric acid. A disadvantage of such an execution is the high material demands with regard to corrosion stability and the separation complexity for recovery and recycling of the homogeneous catalyst.

### Auxiliaries

It has further been found to be advantageous when the bottom of the column contains an inert boiling oil that does not take part in the reaction. Boiling oils in the context of the present invention refer to high-boiling inert substances of prolonged thermal stability. These components have a boiling point higher than the boiling points of the components involved in the reaction. Preference is given to using a boiling oil in order to assure the distillative removal of the (meth)acrylic acid formed without polymerization. However, the boiling point of the boiling oil should not be too high either, in order to reduce the thermal stress on the (meth)acrylic acid formed. More preferably, the boiling temperature of the optionally used boiling oil at standard pressure (1030 mbar) is 170 to 400° C., especially 240 to 290° C.

Suitable boiling oils include relatively long-chain unbranched paraffins having 12 to 20 carbon atoms, aromatic compounds, such as alkyl-substituted phenols or naphthalene compounds, sulfolane (tetrahydrothiophene 1,1-dioxide) or mixtures of these.

Particularly suitable boiling oils have been found here to be 2,6-di-tert-butyl-para-cresol, 2,6-di-tert-butyl-phenol, sulfolane, Diphyl or mixtures of these substances. Most preferably, the boiling oil, which is optional but used with preference, is sulfolane.

Diphyl is a eutectic mixture composed of 75% by weight of biphenyl oxide and 25% by weight of biphenyl.

It has thus also been found to be advantageous to use polymerization inhibitors. The polymerization inhibitors usable with preference include octadecyl 3-(3,5-di-tert-butyl-4-hydroxyphenyl)propionate, phenothiazine, hydroquinone, hydroquinone monomethyl ether, 4-hydroxy-2,2,6,6-tetramethylpiperidinooxyl (TEMPOL), 2,4-dimethyl-6-tert-butylphenol, 2,6-di-tert-butylphenol, 2,6-di-tert-butyl-4-methylphenol, para-substituted phenylenediamines, for example N,N′-diphenyl-p-phenylenediamine, 1,4-benzoquinone, 2,6-di-tert-butyl-alpha-(dimethylamino)-p-cresol, 2,5-di-tert-butylhydroquinone or mixtures of two or more of these stabilizers. Very particular preference is given to phenothiazine and/or hydroquinone monomethyl ether.

The inhibitor can be metered into the feed upstream of the reactor and/or downstream of the reactor and/or into the rectification column, preferably at the top thereof.

### Particular Embodiments

In a particular embodiment of the present invention, the stream from the separation of matter comprising a mixture of MMA and methanol can be processed in a further separate plant in such a way that methanol and MMA are separated from one another. A suitable example for this purpose is a pressure swing distillation. The MMA removed may be returned to the process according to the invention. The methanol may be used in a separate plant for preparation of MMA.

In a particular embodiment of the present invention, the separation of matter of MMA and methanol can be effected by extraction in a separate plant. Especially preferably, this is effected by extraction of the stream obtained via the side draw S1 with the top stream from the rectification column. At least one of the phases formed may be reused here in a chemical reaction. This reaction too may be the production of further alkyl (meth)acrylate, especially of MMA.

Alternatively, it is possible that these separate plants are part of a production plant for preparation of MMA or already exist in another function in these production plants. This production plant may be based on a C2, C3 or C4 process with relatively free choice. A suitable introduction site for the azeotrope of MMA and methanol in C2 and C3 processes is upstream of the phase separation step for the organic and aqueous phases. In C4 processes, introduction upstream of the esterification reaction is suitable. The person skilled in the art is aware of further suitable introduction sites that permit utilization or workup of the azeotrope in the processes described. Skilful coupling of the process according to the invention with processes for preparing MMA can reduce the number of apparatuses, the requirement for auxiliaries and the amount of waste while simultaneously increasing the yield.

### Preferred Executions of the Invention

Three embodiments of the process according to the invention are conceivable for the process. These are shown in FIGS. 1 to 3. The process according to FIG. 1 constitutes the preferred embodiment, since, in this embodiment, the yield and the specific steam consumption can be particularly optimized.

In this illustrative execution, the feed streams of methyl methacrylate (1) and water (2) are mixed with the recycle stream (15) and fed to the preheater (H) and heated to reaction temperature. The recycle stream consists predominantly of the unconverted methyl methacrylate and water reactants, and fractions of methanol and methacrylic acid.

In this embodiment, the reactants (3) heated to reaction temperature are fed to the reactor I (A). The hydrolysis reactor is operated here within a temperature range between 80° C. and 110° C. and within a temperature range between 3 bar and 5 bar. The molar feed ratio of water to methyl methacrylate is between 1.5 and 4. The hydrolysis reaction is preferably executed as a flow tube reactor and is equipped with an acidic fixed bed catalyst.

The reactor product stream (4) is reduced to the column operating pressure by means of expansion valve (B) and fed into the distillation column (C), preferably below the first side draw S1 below the upper separation region (C1) of the column. The column top pressure is between 0.05 and 1 bar. The reactor product stream contains not only the methacrylic acid product but also the methanol reaction by-product and the unconverted methyl methacrylate and water reactants. The distillation column (C) consists of three separation sections: the upper separation section (C1), the middle separation section (C2) and the lower separation section (C3).

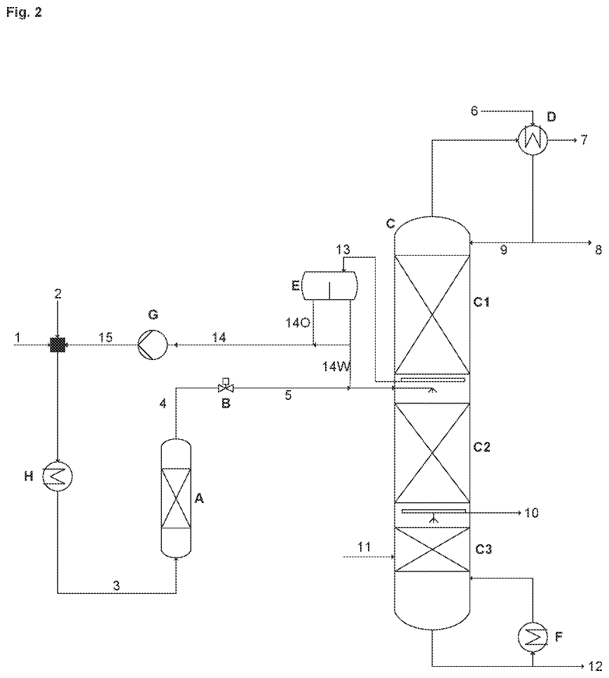

In an alternative embodiment of the present invention, it is optionally possible to install an external phase separator (E) between the upper and middle separation sections according to the above-described first illustrative embodiment. This variant is shown as embodiment II in FIG. 2.

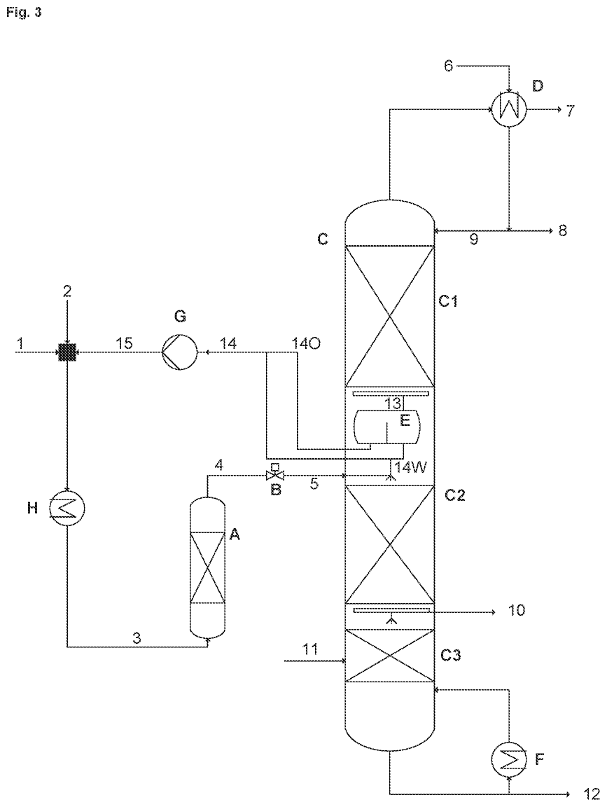

In a further alternative variant, the phase separator (E) may also be installed within the column between the two separation sections (C1) and (C2). This variant is in turn depicted by way of example as embodiment III in FIG. 3.

In the first separation section (C1), the low-boiling reaction by-product methanol is separated from the middle boilers, the water and methacrylic acid reactants, and drawn off at the top. On account of the azeotrope between methanol and methyl methacrylate, the removal of pure methanol is not implementable. Therefore, the overhead product (8) typically contains not only methanol but also methyl methacrylate. The condensation is effected via the condenser (D).

The overhead product (8) can be separated by a workup method suitable for azeotropes into the pure substances methanol and methyl methacrylate. Methyl methacrylate can, for example, be recycled back into the process according to the invention. Alternatively, the skilful introduction of the top product (8) into processes for methyl methacrylate preparation enables the virtually complete recovery of the unconverted methyl methacrylate and the reuse of the methanol by-product. In C2-based processes, the preferred discharge of the azeotropic mixture is into the extraction stage. In C3-based processes, the discharge should be into the distillation column for removal of low boilers. In C4-based processes, a suitable introduction is upstream of the esterification reactors.

In embodiment I—as depicted in FIG. 1—the liquid output stream from the upper separation section (C1) is collected in a collector. This liquid stream is guided out of the column partly or fully as side draw S1 (13). In this case, the second portion is guided as liquid return stream via a distributor to the middle separation section (C2). The side draw (13) is fed to the pump (G).

Alternatively, the liquid output stream (13), in an illustrative embodiment according to FIG. 2, is fed to the phase separator (E). In this case, the organic phase (14O) is removed completely and fed as recycle stream (14) to the pump (G). According to the operating parameters chosen, the aqueous phase (14W) is fed partly or fully as recycle stream (14) to the pump (G). In this case, the second portion is guided as liquid return stream, after mixing with the reactor product (5), as a liquid stream via a distributor to the middle separation section (C2). In this embodiment, the phase separator (E) is outside the column (C).

The phase separator (E) may also be installed within the column, as depicted in illustrative embodiment III according to FIG. 3. In this case, the liquid stream (13) collected in the collector is fed to the phase separator (E). The organic phase (14O) is removed completely and fed as recycle stream (14) to the pump (G). According to the mode of operation, the aqueous phase (14W) is fed partly or fully as recycle stream (14) to the pump (G). The second portion is guided as liquid return stream into a distributor via the middle separation section (C2).

In the middle separation section, in embodiment I, the methacrylic acid product is purified to free it of the water and methyl methacrylate reactants and of traces of methanol that possibly remain. If the process is conducted according to embodiment II or III, in separation section (C2), the methacrylic acid product is purified to free it of methyl methacrylate and traces of methanol and water that possibly remain. The liquid phase from the middle separation section is collected in a collector and sprayed by means of a distributor onto the lower column section (C3).

The gas stream ascending from the lower separation section (C3) is partly discharged from the column as side draw S2 (10) by means of suitable internals. This side stream S2 (10) contains the pure product (methacrylic acid).

In the lower separation section (C3), methacrylic acid is separated from the boiling oil present in the bottom. Stream (11) here is the boiling oil feed stream. The latter is injected via a suitable distributor apparatus, preferably in the upper third of the separation section. The high boilers are drawn off via the bottom draw (12). The high boiler discharge is effected via suitable design of the evaporator (F), for example by means of a thin-film evaporator. Suitable boiling oils are substances having a boiling temperature at standard pressure (1013 mbar) between 200 and 400° C., especially between 240 and 290° C. Suitable boiling oils are described further up.

For avoidance of polymerization, a polymerization inhibitor, which may also be referred to simply as stabilizer, (6) is introduced, preferably at the top of the column. In this regard too, a more detailed description can be found further up.

The side draw S1 (14) is brought to the reactor operating pressure by means of the pump (G) and then mixed with the fresh reactants (1) and (2) as recycle stream (15).

The customary process regime of the prior art according to U.S. Pat. No. 8,791,296 comprises 3 distillation columns in series. In the first column, the azeotrope of methanol and methyl methacrylate is removed. In the second column, methyl methacrylate and water are separated from methacrylic acid, and the variants with or without phase separation constitute possible embodiments at the top. In the third column, methacrylic acid is obtained as distillate. High-boiling by-products are drawn off as bottom product.

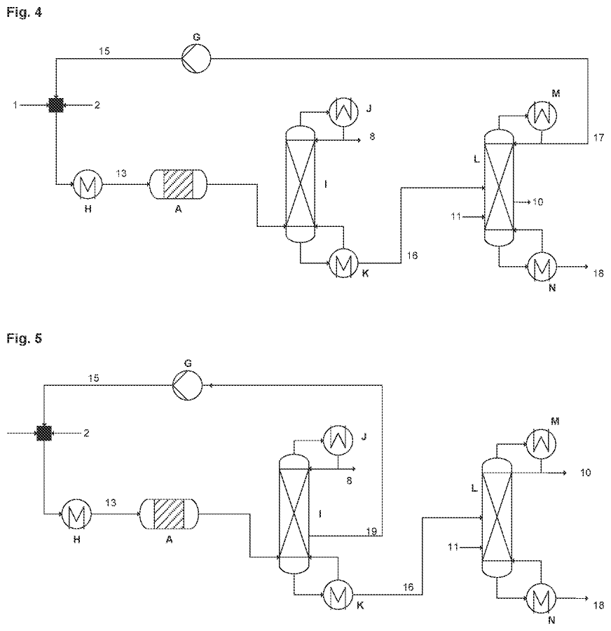

In one embodiment according to FIG. 4, the removal of the azeotrope of methanol and methyl methacrylate (8) which is described in separation section (C1) is first effected in a dedicated distillation column (I), followed by the separation of methyl methacrylate and water (17) from methacrylic acid in a second distillation column (L). Methacrylic acid is drawn off here as side draw (10), and it is optionally possible to use a boiling oil (11) in the bottom for removal of high boilers and reduction of the bottom temperature. Two variants are possible at the top of this second distillation column (L). In a first variant, there is no phase separation, and the condensate is correspondingly divided into reflux and distillate. The second variant has a phase separation at the top. The aqueous phase is used here as reflux and is partly discharged as distillate or from the process. The organic phase is recycled into the reactor as distillate.

In a further interconnection variant (see FIG. 5), the azeotrope of methanol and methyl methacrylate is removed as distillate (8) in the azeotrope column (I). In the same column (I), a mixture of water and methyl methacrylate is removed as side draw (19), and methacrylic acid and high boilers are obtained in the bottom (16). In the MA column (L), pure methacrylic acid is obtained as overhead product (10), and high boilers are removed as bottom product (18). The second column may optionally be operated with boiling oil (11) in order to reduce the bottom temperature.

The processes according to the invention and the alternative embodiments thereof share the features that three separation steps have to be conducted, with the need to separate two azeotropes from the target product. For reduction of the apparatuses required, one or more separation steps are integrated in one apparatus. Preferably, the target product is drawn off here as a side stream. What is also common to these processes is that a heterogeneous catalyst is used for hydrolysis.

As well as the process according to the invention, a plant for preparation of methacrylic acid also forms part of the present invention. This novel plant is characterized in that there is a heterogeneous catalyst for the hydrolysis of methyl methacrylate with water to give methacrylic acid and methanol in a reactor I, and in that the plant, for workup of azeotropes formed from methyl methacrylate and water and from methyl methacrylate and methanol, has a rectification column which has three separation regions and from which methacrylic acid is drawn off from a side draw in high purity.

### LIST OF REFERENCE SYMBOLS

FIG. 1 shows an embodiment without phase separation.

FIG. 2 shows an embodiment with an external phase separator.

FIG. 3 shows an embodiment with an internal phase separator.

FIG. 4 shows an alternative embodiment with two series-connected distillation columns.

FIG. 5 shows a further alternative embodiment with two series-connected distillation columns.

### Streams

(1) Methyl methacrylate feed
(2) Water feed
(3) Reactor feed
(4) Reactor product
(5) Feed to distillation column
(6) Stabilizer addition
(7) Offgas
(8) Distillate (MEOH, MMA)
(9) Distillation column reflux
(10) Side draw S2, MA product stream from separation section C2
(11) Feed stream of boiling oil
(12) Bottom stream
(13) Side draw S1 from separation section C1
(14) Recycle stream at column operation pressure
14W Water phase of the side draw after phase separation
14O Organic phase of the side draw after phase separation
(15) Recycle stream under pressure
(16) Bottom stream from azeotrope column
(17) Distillate from MA column
(18) Bottom stream from MA column
(19) Side draw (MMA & H2O) from azeotrope column

### Apparatuses

(A) Hydrolysis reactor
(B) Expansion valve
(C) Distillation column
   (i) Azeotrope separation section
   (ii) MMA vs. MA separation section
   (iii) MAS vs. boiling oil separation section
(D) Condenser
(E) Decanter
(F) Evaporator
(G) Pump
(H) Preheater
(I) Azeotrope column
(J) Condenser of azeotrope column
(K) Evaporator of azeotrope column
(L) MA column
(M) Condenser of MA column
(N) Evaporator of MA column

### EXAMPLES

### Example 1

In a construction according to the embodiment without phase separation corresponding to FIG. 1, a methyl methacrylate feed stream (1) and a water feed stream (2) are mixed with the recycle stream (15) comprising methanol, water, methyl methacrylate and methacrylic acid. The individual streams have a pressure of 4 bar. The temperature of the streams is 22° C. The methyl methacrylate feed stream (1) is 500 g/h, and the recycle stream (15) is 1539 g/h. The water feed stream (2) is adjusted so as to establish a molar ratio of 2:1 of water to MMA in the mixed overall stream. By means of preheater (H), the stream is heated up to the reaction temperature of 110° C. The result is a dwell time of 60 min in reactor (A), a space-time yield based on methacrylic acid of 200 kg/(h*m³) and a conversion of MMA of 30%. The reactor product stream (4) is expanded to 200 mbar with the aid of expansion valve (B) and guided to the column feed (5) into the distillation column (C). The distillation column is executed as a DN50 glass column. 3 structured packing sections are installed. The uppermost structured packing section (C1) and the middle structured packing section (C2) each have 2 m of Sulzer DX laboratory packing, and the lower structured packing section (C3) has 1 m of Sulzer DX laboratory packing. A collector is installed between the upper and middle structured packing sections, via which the entire liquid phase from the upper section is drawn off as side stream (13). Beneath this collector, the column feed (5) is applied via a distributor to the middle structured packing section (C2). A collector is installed beneath the middle structured packing section, with the aid of which the liquid phase from the middle structured packing section is collected and is guided into a distributor. Between the collector and distributor, there is a stub for removal of the gaseous product stream of methacrylic acid (10). The liquid from the collector is applied to the lower structured packing section (C3) via the distributor. At the top of the column is mounted a condenser (D) that reaches a condensate outlet temperature of 7° C. Evaporator (F) is designed as a thin-film evaporator. The column top pressure is set to 100 mbar. Stabilizer is sprayed by means of conduit (6) onto the condenser for avoidance of polymerization and is guided into the column via the reflux (9). The stabilizer stream has a flow rate of 10 g/h and consists of 2% MEHQ solution in methyl methacrylate. At the top of the column, a reflux ratio of R/D=12 is established. The top temperature of the column is 15.1° C. The upper structured packing section (C1) serves for separation of the azeotrope of methanol and methyl methacrylate from excess methyl methacrylate, water and methacrylic acid. 195 g/h of distillate (8) comprising methanol and methyl methacrylate is drawn off. At the end of the upper structured packing section (C1), 1539 g/h of recycle stream (13) is drawn off as liquid side stream comprising methanol, water, methyl methacrylate and methacrylic acid and fed to the pump (G) and compressed to 4 bar. In the middle structured packing section (C2), 386 g/h of methacrylic acid is separated from the lower-boiling methyl methacrylate and water components and drawn off as gaseous side stream (10). In the lower structured packing section (C3), in the middle, 10 g/h of sulfolane (11) is applied to the column as boiling oil. This achieves the effect that methacrylic acid is not subjected to temperatures higher than 95° C., which reduces the polymerization risk. At the same time, high boilers formed are discharged as bottom stream (12) via the thin-film evaporator (F). The bottom temperature is 198° C. Also produced is a virtually methacrylic acid-free bottom phase, which minimizes methacrylic acid losses. Table 1 lists the mass flow rates observed and the physical composition of the individual streams.

TABLE 1
Mass flow rates and physical composition

| | | (1) | (2) | (3) | (4) | (6) | (7) | (8) | (10) |
|---|---|---|---|---|---|---|---|---|---|
| Mass flow rate | g/h | 500 | 81 | 2121 | 2121 | 10 | 0 | 195 | 386 |
| MEOH | % by wt. | | | 5.5 | 12.3 | | | 73.7 | |
| H2O | % by wt. | | 100.0 | 24.6 | 20.8 | | | | traces |
| MMA | % by wt. | 100.0 | | 68.2 | 47.1 | 98.0 | | 26.3 | |
| MAA | % by wt. | | | 1.7 | 19.9 | | | | 99.9 |
| Sulfolane | % by wt. | | | | | | | | |
| MEHQ | % by wt. | | | | | 2.0 | | traces | traces |

| | | (11) | (12) | (13) |
|---|---|---|---|---|
| Mass flow rate | g/h | 10 | 10 | 1539 |
| MEOH | % by wt. | | | 7.6 |
| H2O | % by wt. | | | 28.6 |
| MMA | % by wt. | | | 61.6 |
| MAA | % by wt. | | traces | 2.3 |
| Sulfolane | % by wt. | 100 | 99.9 | |
| MEHQ | % by wt. | | traces | traces |

Table 2 shows the specific auxiliary consumptions achieved by the process.

| | Steam | Cooling brine |
|---|---|---|
| | kgₛₜₑₐₘ/kg_{MA} | P_{brine}/kg_{MA} |
| Preheater (H) | 0.72 | |
| Distillation column (C) | 3.75 | 312 |
| Total | 4.47 | 312 |

A molar yield of 0.90 mol of MA per mole of MMA used is achieved.

### Example 2

In a construction according to the embodiment with external phase separation corresponding to FIG. 2, a methyl methacrylate feed stream (1) and a water feed stream (2) are mixed with the recycle stream (15) comprising methanol, water, methyl methacrylate and methacrylic acid. The individual streams have a pressure of 4 bar. The temperature of the streams is 22° C. The methyl methacrylate feed stream (1) is 500 g/h, and the recycle stream (15) is 1353 g/h. The water feed stream (2) is adjusted so as to establish a molar ratio of 2:1 of water to methyl methacrylate in the mixed overall stream. By means of preheater (H), the stream is heated up to the reaction temperature of 110° C. The result is a dwell time of 60 min in reactor (A), a space-time yield based on methacrylic acid of 200 kg/(h*m³) and a conversion of MMA of 30%. The reactor product stream (4) is expanded to 200 mbar with the aid of expansion valve (B) and guided to the column feed (5) into the distillation column (C). The distillation column is executed as a DN50 glass column. 3 structured packing sections are installed. The uppermost structured packing section (C1) and the middle structured packing section (C2) each have 2 m of Sulzer DX laboratory packing, and the lower structured packing section (C3) has 1 m of Sulzer DX laboratory packing. A collector is installed between the upper and middle structured packing sections, via which the entire liquid phase from the upper structured packing section (C1) is drawn off as side stream (13). The column feed (5) is applied via a distributor to the middle structured packing section (C2). A collector is installed beneath the middle structured packing section, with the aid of which the liquid phase from the middle structured packing section is collected and is guided into a distributor. Between the collector and distributor, there is a stub for removal of the gaseous product stream of methacrylic acid (10). The liquid from the collector is applied to the lower structured packing section (C3) via the distributor. At the top of the column is installed a condenser (D) that reaches a condensate outlet temperature of 7° C. Evaporator (F) is designed as a thin-film evaporator. The column top pressure is set to 100 mbar. Stabilizer is sprayed by means of conduit (6) onto the condenser for avoidance of polymerization and is guided into the column via the reflux (9). The stabilizer stream has a flow rate of 10 g/h and consists of 2% MEHQ solution in methyl methacrylate. At the top of the column, a reflux ratio of R/D=12 is established. The top temperature of the column is 15.9° C. The upper structured packing section (C1) serves for separation of the azeotrope of methanol and methyl methacrylate from excess methyl methacrylate, water and methacrylic acid. 219 g/h of distillate (8) comprising methanol and methyl methacrylate is drawn off. At the end of the upper structured packing section (C1), 1776 g/h of liquid phase is drawn off as side stream (13) comprising methanol, water, methyl methacrylate and methacrylic acid and fed to the phase separator (E). This forms 846 g/h of aqueous phase (14W) and 930 g/h of organic phase (14O). The aqueous phase (14W) is divided in a ratio of 1:1, with mixing of the first portion with the column feed (15) and application to the middle structured packing section (C2) via a distributor. The second portion is mixed with the organic phase (14O) and results in the recycle stream (14) in an amount of 1353 g/h. This stream is compressed to 4 bar by means of pump (G). In the middle structured packing section (C2), 358 g/h of methacrylic acid is separated from the lower-boiling methyl methacrylate and water components and drawn off as gaseous side stream (10) below the collector below the middle structured packing section (C2). In the lower structured packing section (C3), in the middle, 10 g/h of sulfolane (11) is applied to the column as boiling oil. This achieves the effect that methacrylic acid is not subjected to temperatures higher than 95° C., which reduces the polymerization risk. At the same time, high boilers formed are discharged as bottom stream (12) via the thin-film evaporator (F). The bottom temperature is 198° C. Also produced is a virtually methacrylic acid-free bottom phase, which minimizes methacrylic acid losses. Table 3 lists the mass flow rates observed and the physical composition of the individual streams.

TABLE 3
Mass flow rates and physical composition

| | | (1) | (2) | (3) | (4) | (6) | (7) | (8) | (10) |
|---|---|---|---|---|---|---|---|---|---|
| Mass flow rate | g/h | 500 | 77 | 1930 | 1930 | 10 | 0 | 219 | 358 |
| MEOH | % by wt. | | | 1.4 | 8.2 | | | 60.6 | |
| H2O | % by wt. | | 100.0 | 25.0 | 21.1 | | | 0.9 | traces |
| MMA | % by wt. | 100.0 | | 69.4 | 47.9 | 98.0 | | 38.5 | |
| MAA | % by wt. | | | 4.2 | 22.7 | | | | 99.9 |
| Sulfolane | % by wt. | | | | | | | | |
| MEHQ | % by wt. | | | | | 2.0 | | traces | traces |

| | | (11) | (12) | (13) | (14) | (14O) | (14W) |
|---|---|---|---|---|---|---|---|
| Mass flow rate | g/h | 10 | 10 | 1776 | 1353 | 930 | 846 |
| MEOH | % by wt. | | | 2.5 | 1.9 | 0.8 | 4.5 |
| H2O | % by wt. | | | 44.7 | 30.0 | 1.9 | 91.7 |
| MMA | % by wt. | | | 47.8 | 62.1 | 89.3 | 2.2 |
| MAA | % by wt. | | traces | 5.0 | 6.0 | 8.0 | 1.6 |
| Sulfolane | % by wt. | 100 | 99.9 | | | | |
| MEHQ | % by wt. | | traces | traces | traces | traces | traces |

Table 4 shows the specific auxiliary consumptions achieved by the process.

| | Steam | Cooling brine |
|---|---|---|
| | kgₛₜₑₐₘ/kg_{MA} | P_{brine}/kg_{MA} |
| Preheater (H) | 0.67 | |
| Distillation column (C) | 4.14 | 346 |
| Total | 4.81 | 346 |

A molar yield of 0.83 mol of MA per mole of MMA used is achieved.

### Comparative Example 3

A construction according to publication U.S. Pat. No. 8,791,296, consisting of three DN50 glass distillation columns each with 2 m of Sulzer DX laboratory packing, a flow tube reactor and the appropriate auxiliary apparatuses, for example heat exchanger, evaporator and pumps, is supplied with a methyl methacrylate feed stream of 500 g/h and a water feed stream of 82 g/h. These reactant streams are mixed with the recycle stream (1471 g/h), which is the overhead product from the second distillation column, and form the reactor feed stream of 2052 g/h. The water feed stream was adjusted so as to establish a molar ratio of 2:1 of water to methyl methacrylate in the reactor feed stream. The individual streams have a pressure of 4 bar. By means of a preheater, the reactor feed stream is heated up to the reaction temperature of 110° C. The result is a dwell time of 60 min in reactor (A), a space-time yield based on methacrylic acid of 200 kg/(h*m³) and a conversion of MMA of 30%. The reactor product stream contains methanol, water, methyl methacrylate and methacrylic acid, and is guided into the bottom of the first distillation column. At a top pressure of 1000 mbar, a top temperature of 64.3° C. and a bottom temperature of 83.3° C. are established. The reflux ratio is set to 12. At the top, the azeotrope (196 g/h) consisting of MeOH and MMA is drawn off. The bottom stream is 1840 g/h and comprises predominantly water, methyl methacrylate and methacrylic acid, and a little methanol.

This stream is guided into the middle of a downstream second distillation column which is operated at a top pressure of 100 mbar. A top temperature of 38.9° C. and a bottom temperature of 93.2° C. are established. The reflux ratio is 0.7. The overhead product (1471 g/h) consists of the azeotrope of water and MMA. The bottom product contains MA and traces of high boilers and stabilizers and is 385 g/h.

The bottom product from the second distillation column is fed into he middle of the third distillation column for fine purification of the methacrylic acid. The third column is operated at a top pressure of 100 mbar, and a top temperature of 93.2° C. and a bottom temperature of 98.6° C. are established. The reflux ratio is set to 2. At the top, 380 g/h of methacrylic acid is obtained as pure product. In the bottom, 5 g/h of high boilers and stabilizers is removed via a thin-film evaporator.

Each of the three distillation columns has a stabilizer addition at the condenser for avoidance of polymerization, and this reaches the columns via the reflux. The stabilizer stream for each column has a flow rate of 10 g/h and consists of 2% MEHQ solution in methyl methacrylate. Table 5 lists the mass flow rates observed and the physical composition of the individual streams.

TABLE 5
Mass flow rates and physical composition

| | | | | Reactor | Reactor | Column 1 |
|---|---|---|---|---|---|---|
| | | MMA feed | H2O feed | feed | product | distillate |
| Mass flow rate | g/h | 500 | 82 | 2052 | 2052 | 196 |
| MEOH | % by wt. | | | 2.5 | 9.5 | 73.0 |
| H2O | % by wt. | | 100.0 | 25.3 | 21.4 | 0.4 |
| MMA | % by wt. | 100.0 | | 70.4 | 48.6 | 26.6 |
| MAA | % by wt. | | | 1.8 | 20.5 | |
| MEHQ | % by wt. | | | traces | traces | traces |

| | | Column 1 | Column 2 | Column 2 | Column 3 | Column 3 |
|---|---|---|---|---|---|---|
| | | bottom | distillate | bottom | distillate | bottom |
| Mass flow rate | g/h | 1856 | 1471 | 385 | 380 | |
| MEOH | % by wt. | 2.8 | 3.5 | | | |
| H2O | % by wt. | 23.6 | 29.8 | traces | traces | |
| MMA | % by wt. | 50.9 | 64.2 | | | |
| MAA | % by wt. | 22.7 | 2.5 | 99.9 | 99.9 | 95.0 |
| MEHQ | % by wt. | traces | traces | traces | | traces |

Table 6 shows the specific auxiliary consumptions achieved by the process.

| | Steam | Cooling brine |
|---|---|---|
| | kgₛₜₑₐₘ/kg_{MA} | P_{brine}/kg_{MA} |
| Preheater | 0.67 | |
| Distillation column 1 | 2.56 | 160 |
| Distillation column 2 | 2.88 | 186 |
| Distillation column 3 | 0.63 | 36 |
| Total | 6.74 | 382 |

A molar yield of 0.88 mol of MA per mole of MMA used is achieved.

1-17: (canceled)

18: A process for continuously preparing (meth)acrylic acid by reacting (meth)acrylic esters with water, the process comprising:
(a) in a reactor I, reacting a (meth)acrylic ester and water in the presence of a Brønsted acid, to obtain a mixture comprising the (meth)acrylic ester, water, an alcohol corresponding to the (meth)acrylic ester, and an unsaturated acid,
(b) separating the mixture in a rectification column having an upper, middle and lower region, such that
(i) a column distillate removed in the upper region of the rectification column is a mixture consisting of the alcohol and the (meth)acrylic ester,
(ii) a mixture of the (meth)acrylic ester and water is removed in a side draw S1 of the rectification column,
(iii) (meth)acrylic acid is removed in a side draw S2 of the rectification column, and
(iv) a substance mixture comprising higher-boiling components compared to (meth)acrylic acid is removed in the lower region, in the bottom of the rectification column.

19: The process according to claim 18, wherein the (meth)acrylic ester and water starting materials present in the side draw S1 are returned to a reaction region of the reactor I, where they are reacted together with fresh water and (meth)acrylic ester and, optionally, the side draw stream is subjected to a phase separation before being at least partly recycled into the reaction.

20: The process according to claim 18, wherein the (meth)acrylic acid is removed in a purity greater than 95% by weight via the side draw S2, and the side draw S2 is beneath the side draw S1 in the rectification column.

21: The process according to claim 18, wherein the bottom of the rectification column contains an inert boiling oil that does not take part in the reaction.

22: The process according to claim 18, wherein reactor I is supplied continuously with a reactant mixture of the (meth)acrylic ester and water in a molar ratio between 1:20 and 20:1.

23: The process according to claim 18, wherein the Brønsted acid in reactor I is a heterogeneous acidic fixed bed catalyst.

24: The process according to claim 23, wherein an acidic cation exchanger is used as catalyst.

25: The process according to claim 18, wherein the reactor I is outside the rectification column.

26: The process according to claim 18, wherein the (meth)acrylic acid is methacrylic acid, the (meth)acrylic ester is methyl methacrylate, and the alcohol is methanol.

27: The process according to claim 21, wherein the boiling oil used is a high-boiling inert substance having a boiling point higher than boiling points of components involved in the reaction.

28: The process according to claim 27, wherein the boiling oil used is 2,6-di-tert-butyl-para-cresol, 2,6-di-tert-butyl-phenol, sulfolane, diphyl, or a mixture of these substances.

29: The process according to claim 18, wherein high-boiling components are discharged from the bottom of the rectification column, and evaporating substances are recycled into the rectification column.

30: The process according to claim 26, wherein the top stream from the rectification column is subjected to a further separation of matter in order to obtain methyl methacrylate and methanol.

31: The process according to claim 30, wherein a stream from the further separation of matter comprising a mixture of MMA and methanol is processed in a further separate plant in such a way that the methanol and the MMA are separated from one another, and wherein the further separate plant is a plant for preparation of MMA based on a C2, C3, or C4 process, with at least partial conversion of isolated methanol to further MMA in the further separate plant.

32: The process according to claim 30, wherein the further separation of matter of MMA and methanol is an extractive separation, with reuse of at least one of the phases formed in a chemical reaction.

33: A plant for preparation of methacrylic acid, comprising a heterogeneous catalyst for hydrolysis of methyl methacrylate with water to give methacrylic acid and methanol in a reactor I, and
wherein the plant, for workup of azeotropes formed from methyl methacrylate and water and from methyl methacrylate and methanol, has a rectification column which has three separation regions and from which methacrylic acid is drawn off from a side draw in high purity.

34: The process according to claim 28, wherein the boiling oil is sulfolane.

35: The process according to claim 32, wherein the extractive separation is by extraction of the stream obtained via side draw S1 with the top stream from the rectification column.
